(19) United States
(12) Patent Application Publication
(10) Pub. No.: US 2023/0338590 A1
(43) Pub. Date: Oct. 26, 2023

(54) MATERIALS AND METHODS FOR TREATING CORONAVIRUS

(71) Applicant: MONOPAR THERAPEUTICS, INC., Wilmette, IL (US)

(72) Inventors: Andrew P. Mazar, Lake Forest, IL (US); James T. Harvey, Naperville, IL (US)

(21) Appl. No.: 18/010,510

(22) PCT Filed: Jun. 15, 2021
(86) PCT No.: PCT/US21/37416

§ 371 Date: Dec. 15, 2022

Related U.S. Application Data
(60) Provisional application No. 63/039,299, filed on Jun. 15, 2020.

(51) Int. Cl. A61K 51/10

(52) U.S. Cl. CPC A61K 51/1075; A61K 51/1093; A61K 2121/00

(57) ABSTRACT

Antibodies specific for uPAR and uPA-uPAR complexes in the form of radioconjugates with alpha particle-emitting radionuclides are disclosed as is their use in treating severe respiratory diseases such as severe COVID-19.

### CROSS-REFERENCE TO RELATED APPLICATIONS

The present application claims the priority benefit of U.S. Provisional Patent Application No. 63/039,299 filed, Jun. 15, 2020, herein incorporated by reference in its entirety.

### FIELD OF THE INVENTION

The present invention in the field of biochemistry, immunology and medicine relates to antibodies (“Abs”) specific the urokinase plasminogen activator receptor (uPAR), conjugated to alpha emitting radionuclides for treatment of severe COVID-19 by selective damage to highly activated myeloid cells in the lungs and elsewhere.

### BACKGROUND

A significant body of evidence from studies in vitro and in vivo has established that the urokinase plasminogen activator (uPA) system is central to the process of metastasis, making it a promising target for cancer drug development (Mazar, A P et al. (1999) Angiogenesis 3: 15-32). In addition to uPA, its cell surface receptor (uPAR) is a suitable target for the design and development of cancer therapeutic and diagnostic agents (Mazar, A P (2001) Anti-Cancer Drugs 12: 397-400) because:
(a) uPAR is selectively expressed on metastatic tumor cells and angiogenic endothelial cells (“ECs”), hyperstimulated myeloid inflammatory cells, but not on most other normal cells;
(b) uPAR is an important participant in several extracellular and intracellular pathways required for metastasis that are currently the object of intense drug development efforts; and
(c) it is possible to interfere at several different points along the uPA pathway.
   Thus, uPA and uPAR are promising targets for the development of diagnostics and therapeutics useful against many different types of tumors/cancers as well as other diseases in which uPAR is highly expressed on pathogenic cells.

uPA/uPAR in Hyperinflammation

The urokinase system including the ligand, uPA and its receptor, uPAR, are hallmarks of the activation of myeloid cells that cause hyperinflammation and are found expressed on activated macrophages, neutrophils and fibroblasts in acute lung injury and other organ injury in models of acute respiratory distress syndrome (ARDS) but not in normal adult tissue (Mazar A P et al., Curr Pharm Des. 2011; 17: 1970-8; Marudamuthu A S et al., J Biol Chem. 2015; 290:9428-41).

Immune cells of the lymphoid lineage to do not typically express uPAR (Mazar et al., supra). A soluble form of uPAR (suPAR) that circulates and can be measured in plasma is shed from the surface of neutrophils and macrophages in ARDS and correlates with increased hyperimmune response mediated by these cells ((Gussen H et al., J Intensive Care. 2019, 7:26; Liu G et al., PLoS One; 6: e25843; Ni W et al., Sci Rep. 2016, 6: 39481). suPAR levels correlated with the severity, inflammation and mortality from ARDS and are prognostic for the development of ARDS in patients with sepsis (Chen D et al., Exp Ther Med. 2019; 18: 2984-29920). suPAR is also prognostic for mortality in intensive care units in ARDS patients (Geboers D G et al., Intensive Care Med. 2015; 41:1281-90)) and a marker of infection in patients who develop acute kidney injury from the infection (Hall A et al., BMC Nephrol. 2018; 19: 191). Finally, a study just published demonstrates that suPAR is an early predictor of severe respiratory failure in COVID-19 (Rovina N et al., Crit Care. 2020; 24:1870). In this study, neutrophilia also correlates with the development of COVID-19 respiratory distress. Targeting uPAR (as described herein) could lead to the rapid depletion of activated myeloid cells and would attenuate the cytokine storm and its sequelae (ARDS, coagulopathy, decreased T cells).

Despite the promise of targeting the uPA system for therapeutic and diagnostic purposes, research efforts have not resulted in the development of clinically suitable agents. Small molecule approaches have been hampered by (1) the difficulty of potently inhibiting a protein-protein interaction (e.g., uPA-uPAR or uPAR-integrin), and (2) the lack of suitable leads or structural information amenable to medicinal chemistry efforts.

Severe COVID-19 Disease

Infection with SARS-CoV2, a novel coronavirus, causes COVID-19 illness. The most common symptoms at onset of COVID-19 are fever, cough, and fatigue, similar to other respiratory viruses, but COVID-19 has unique manifestations including loss of taste and smell, headache, sore throat, and gastrointestinal symptoms such as nausea, vomiting and diarrhea. Two distinct but overlapping pathological subsets of COVID-19 patients whose symptoms can be classified as follows:
(1) those triggered by the virus itself and
(2) those triggered by the host response.

The majority of patients fall into the first subset of patients who exhibit some viral replication and have mild to moderate symptoms that do not require hospitalization (Rothan H A et al., J Autoimmun. 2020: 102433). The innate immune response to SARS-Cov2 in these patients limits the extent of the disease and they recover spontaneously with little or no medical intervention. However, the second subset of patients (up to 31% of patients based on a recent study, England J T et al., Blood Rev. 2020 15:100707) develop severe COVID-19 disease that is frequently characterized by the development of viral pneumonia and severe respiratory distress requiring hospitalization and oxygen support. Patients that progress to this severe acute respiratory syndrome (SARS/ARDS) often require ventilator support and a majority of them die from the sequelae of SARS/ARDS including systemic inflammation related to a “cytokine storm” that leads to multi-organ failure.

COVID-19 patients that present with pneumonia may also experience a number of abnormally critical features associated with SARS/ARDS local and systemic inflammation, including ground-glass lung opacities and patchy consolidation; cardiac and kidney damage; and coagulopathies characterized by lung embolism and stroke, which often lead to death (Rothan et al., supra). The host response in these severe COVID-19 patients becomes progressively abnormal and is characterized by higher leukocyte numbers, abnormal respiratory findings including hypoxia, and increased systemic levels of circulating pro-inflammatory cytokines including IL1-β, IL-6, G-CSF, TNFα, and many others (Zhou G et al., Front Med. 2020; 14: 117-125). Several recently published studies that followed severe COVID-19 patients with SARS/ARDS indicated a hyperstimulated immune system with both systemic and local inflammatory responses based on systemic cytokines as well as the appearance of markers of heart and kidney damage (Cao, X, Nat Rev Immunol 2020, 20: 269-270). Paradoxically, patients that progress to severe COVID-19 have progressively lower levels of the T lymphocytes typically mobilized to fight viral infections and regulate inflammatory responses, and much higher levels of myeloid inflammatory cells such as monocytes/macrophages and neutrophils which are a product of a hyperstimulated immune system; these rogue myeloid cells that produce high systemic levels of cytokines (the cytokine storm), are the major cause of the respiratory and multi-organ failure seen in patients that die from COVID-19.

A cytokine storm is an uncontrolled systemic inflammatory response to SARS-CoV2 caused by hyperstimulated myeloid cells that affect multiple organs. Patients experiencing a cytokine storm can rapidly develop a broad range of sequelae from the uncontrolled release of pro-inflammatory cytokines leading to damage in the lung, kidney, heart and brain, and coagulopathies characterized by the formation of blood clots, that can lead to stroke and myocardial infarction (MI) (FIG. 1).

These patients require rapid intervention. Attenuation of the cytokine response is thought to be key to minimizing the damage that occurs in response to this hyperstimulation of the innate immune response. Patients with severe COVID-19 undergoing rapid clinical decline often exhibit high virus titers and cytokine storms that correlate with the morbidity and mortality observed in severe COVID-19 (An P J et al., Pharmacol Res. 2020 May 22:104946).

Cytokine storms are characterized by several processes that drive organ damage and failure. A “normal” myeloid and T cell response to a virus would mediate virus clearance, clearance of virus-infected cells and would limit further damage to host by controlling the response (Kim K D et al. Nat Med 2007; 13: 1248-1252; Palm N W and Medzhitov, R., Nat Med 2007; 13: 1142-1144). However, a cytokine storm caused by pathogenic SARS-CoV2 results in a diminished T cell response, potentially via TNF-mediated T cell apoptosis, and inhibition of macrophages polarization to an anti-inflammatory phenotype all of which contribute to the uncontrolled inflammatory response (Channappanavar R et al., Sem Immunopathol. 2017; 39: 529-539). Furthermore, tissue homeostasis is altered, leading to organizing lung injury characterized by fibrosis and infiltration of activated macrophages and neutrophils. In mouse models of ARDS and of BARS-CoV infection, these investigators found enhanced perivascular infiltration of alternatively activated macrophages, neutrophils, and fibroblasts accompanied by extensive fibrin deposition and alveolar collapse, features observed during end stage ARDS in humans. These results suggest that novel strategies directed at attenuating the hyperinflammatory responses will improve clinical outcomes for patients with severe COVID-19.

The correlation between increased cytokines, decreased T cells and poor outcomes in COVID-19 patients is supported by the recent study of Diao et al. (Front Immunol. 2020, 11:827). This study retrospectively reviewed total T cell counts, CD4+/CD8+ T cell subsets, and serum cytokine concentrations from 522 in-patients with laboratory-confirmed COVID-19 in Wuhan, China, admitted to two hospitals from December 2019 to January 2020. These patients were compared to 40 healthy controls. The study also measured the expression of T cell exhaustion markers PD-1 and Tim-3 in the peripheral blood of 14 COVID-19 cases. The number of total T cells, CD4+ and CD8+ T cells were dramatically reduced in COVID-19 patients, especially among elderly patients (≥60 years of age) and in patients admitted to the ICU. Low T cells, CD4+ T cells or CD8+ T cell counts were negatively correlated with patient survival and serum IL-6, IL-10 and TNF-α concentration. Patients who improved demonstrated reduced IL-6, IL-10 and TNF-α and restored T cell counts. Finally, T cells from COVID-19 patients expressed significantly higher levels of the T cell exhaustion marker PD-1 compared to healthy controls, and increasing PD-1 expression on T cells could be seen as patients progressed from prodromal to overtly symptomatic stages, further indicating T cell exhaustion.

There are currently no approved therapeutic interventions for patients with severe COVID-19, and the majority of patients who become hypoxic are managed by supportive measures such as oxygen support, mechanical ventilation or extracorporeal ventilation. Unfortunately, several recent studies demonstrated that the mortality in severe COVID-19 patients that are placed on ventilators support is >60% (Yang X et al, Lancet Respir Med. 2020; 8: 475-481; Richardson S et JAMA. 2020, 323: 2052-9). Even in patients that survive severe COVID-19, recovery times are long and patients experience post-ICU syndrome and long term neurological and physical complications that require significant rehabilitation (12).

In the absence of a vaccine or other therapeutic options to treat patients who become infected, concerns about viral transmission and the possibility of developing severe COVID-19 interferes with societal and economic recovery. The development of a therapeutic intervention that treats severe COVID-19 will mitigate many of the concerns that currently dictate the requirements for social distancing, masking and limiting interactions to small groups and the resultant negative effect on societies and economies around the world. There would be less concern about COVID-19 transmission and illness if therapeutic options existed. Furthermore, treatments for severe COVID-19 will be needed regardless of whether a vaccine is developed that prevents COVID-19 in a majority of populations. In the paradigm of other respiratory illnesses such as influenza or pneumonia where effective vaccines exist, there is always s subset of patients in whom the vaccine is not protective. Some of these patients develop fatal severe respiratory distress from influenza or pneumonia each year. For example, there have been >80,000 deaths from pneumonia and influenza in the 2019-2020 flu season (CDC, 2020). Thus, the expectation is that while a SARS-Cov2 vaccine will help prevent many infections, some infections will still occur and some of these may be severe and require a therapeutic intervention that prevents mortality. Similarly, since similar host hyperimmune responses leading to SARS/ARDS observed in severe COVID-19 patients are also observed in patients with other respiratory virus infections (such as influenza, pneumonia and other coronaviruses such as SARS-CoV), the therapeutic intervention described herein should be useful to treat SARS/ARDS regardless of the pathogen and may be used more broadly beyond severe COVID-19.

### SUMMARY OF THE INVENTION

The present invention is directed to development of a uPAR-targeted precision radioimmunotherapeutic (“uPRIT”) agent which is a radioconjugate that targets and attenuates hyperactivated myeloid cell number and functions for treatment of severe COVID-19 and to methods using such a therapeutic to reduce the risk of death and improve the long-term outcomes in patients with severe respiratory distress, e.g., from COVID-19 infection.

One of the present inventors and colleagues produced monoclonal antibodies (mAbs) that bind to uPA-uPAR complexes and that inhibit their interaction of with downstream targets (such as integrins). See: U.S. Pat. Nos. 8,101,726 and 8,105,602 which are incorporated by reference in their entirety. Such inhibition of uPA-uPAR interaction inhibited tumor growth and metastasis and is expected to be the basis for a radioimmunotherapeutic composition and method to treat severe COVID-19 disease, reducing both mortality and morbidity.

While these mAbs showed utility as “naked” antibodies, the present invention focuses on their ability to target therapeutic agents, preferably therapeutic radionuclides (radioconjugates), to sites of immunopathology in COVID-19 disease.

The epitopes recognized by these mAbs are peptide regions within uPAR. Therefore uPAR peptides corresponding to these regions or derived therefrom are useful as antagonists of uPAR interactions with downstream proteins. Exemplary uPAR peptides are described herein.

Thus, mAbs that target and inhibit uPA-uPAR interactions with downstream targets or kill uPAR-expressing cells are useful not only in the treatment and/or diagnosis of cancer but in treatment of severe respiratory distress, e.g., from COVID-19 disease Preferred downstream ligands of uPA-uPAR, or of uPAR alone, include integrins, low-density lipoprotein receptor-related protein (LRP) as well as other binding partners. Some of these downstream ligands may mediate cell signaling, migration and/or invasion into tissue.

The present inventors have produced and studied two monoclonal antibodies, MNPR-101 and MNPR-102 that specifically bind ligand-occupied uPAR and thus serve as exemplary molecules that can bind uPAR regardless of the presence of ligand. The monoclonal antibodies can detect both occupied and unoccupied uPAR in a tumor or other diseased tissue where the uPA system plays a role in the pathobiology. Preferred antibodies or other non-Ab-like ligands are those that do not bind to the uPA-binding site of uPAR.

In addition, the present inventors have developed a method to identify antibodies that mimic the characteristics of MNPR-101 and MNPR-102. This method can be used to develop humanized or fully human mAbs that recognize and bind to the same epitopes as those bound by MNPR-101 and MNPR-102. Such mimics, the former of which has particularly robust anti-tumor activity and binding to and actions on myeloid inflammatory cells, are included herein as therapeutic and/or diagnostic agents.

The present invention is further directed to macromolecules, including Abs, antigen binding fragments such as single chain Abs (such as scFv), non-Ab polypeptides and peptides, aptamers, etc., as well as small organic molecules, that have the property of binding to uPAR without inhibiting the binding of uPA. Some of these molecules interfere with downstream interactions of either uPA-uPAR or uPAR alone.

In one embodiment, a preferred uPRIT comprises a mAb or antigen-binding fragment that comprises:
(a) a V_{L} chain comprising three CDR's which have the respective amino acid sequences SEQ ID NO:3, SEQ ID NO:4 and SEQ ID NO:5; and
(b) a V_{H} chain comprising three CDR's which have the respective amino acid sequences SEQ ID NO:6, SEQ ID NO:7 and SEQ ID NO:8.

A more preferred mAb component of the uPRIT comprises
(a) a V_{L} chain with the sequence SEQ ID NO:1; and
(b) a V_{H} chain with the sequence SEQ ID NO:2.

In another preferred embodiment, the mAb or antigen-binding fragment component of the uPRIT comprises:
(a) a V_{L} chain comprising three CDR's which have the respective amino acid sequences SEQ ID NO:11, SEQ ID NO:12 and SEQ ID NO:13; and
(b) a V_{H} chain comprising three CDR's which have the respective amino acid sequences SEQ ID NO:14, SEQ ID NO:15 and SEQ ID NO:16.

A more preferred mAb or antigen binding fragment component of the uPRIT comprises:
(a) a V_{L} chain having the sequence set out in SEQ ID NO:9; and
(b) a V_{H} chain having the sequence set out in SEQ ID NO:10.

In a preferred uPRIT, the mAb is (a) one designated MNPR-101 produced by a hybridoma having ATCC Accession #PTA-8191. Another is the mAb designated MNPR-102 (previously designated ATN-615) produced by a hybridoma having ATCC Accession #PTA-8192. Most preferred are humanized versions of these mAbs.

One of the present inventors and colleagues identified the epitopes to which these Abs bind. See in in particular, U.S. Pat. No. 8,105,602. Those peptides include epitopes, particularly conformation-dependent epitopes, defined by the amino acid sequences shown in Table 1.

TABLE 1
uPAR epitope sequences

| | Amino acids of | SEQ ID |
|---|---|---|
| Sequence | Human uPAR¹ | NO: |
| CCTKSGCNHPDLDVQYPSG | 265-283 | 17 |
| CCTKSGCNHPDLDVQYPS | 265-282 | 18 |
| CCTKSGCNHPDLDVQYP | 265-281 | 19 |
| CCTKSGCNHPDLDVQY | 265-280 | 20 |
| CCTKSGCNHPDLDVQ | 265-279 | 21 |
| CGSSDMSCERGRHQSL | 98-114 | 22 |
| KSGCNHPDLD | 268-277 | 23 |

¹The amino acid numbering reflects the processed form of uPAR

Also included is a uPRIT comprising a mAb having essentially the same antigen-binding characteristics as MNPR-101 and a mAb having essentially the same antigen-binding characteristics as MNPR-102.

In a preferred embodiment, as described herein, the mAb or antigen binding fragment is conjugated to an appropriate alpha emitting radionuclide to form uPRIT. Also included are “diagnostically labeled” conjugates in which a detectable label is conjugated to the above mAb. Alpha emitters include direct alpha emitters and alpha generators (such as ²¹²Pb) that are not direct alpha emitters but decay to a high energy alpha emitter in patients.

Preferred alpha emitting radioisotopes conjugated to the mAb in the present invention are presented below. These then serve as therapeutic pharmaceutical compositions that treat severe respiratory distress, such as COVID-19 disease. The therapeutically active radioisotope may be conjugated directly to, or bound indirectly to, the mAb. Examples of various therapeutic radionuclide useful herein, but not limited to, include ⁴⁷Sc, ⁹⁰Y, ¹⁰⁹Pd, ¹²⁵I, ¹³¹I, ¹⁸⁶Re, ¹⁸⁸Re, ¹⁹⁹Au, ²¹¹At, ²¹²Pb, ²¹³Bi, ²²³Ra, ²²⁷Th, or ²²⁵Ac.

Also included in this invention is a method for contacting myeloid or other uPAR-expressing immune cells with uPRIT to inhibit their activity and to induce apoptosis or necrosis in these cells.

Also included is a method for treating a subject having a disease, disorder or condition characterized by undesired hyperstimulated myeloid inflammation comprising administering to the subject an effective amount of the above therapeutic pharmaceutical composition.

The invention includes an assay method for identifying an Ab or other ligand that binds to the same epitope as does mAb MNPR-101 or mAb MNPR-102 or comprising measuring the ability of a sample suspected of containing the Ab or other ligand to competitively inhibit the binding of detectably labeled MNPR-101 or MNPR-102 to (i) immobilized suPAR, (ii) immobilized suPAR D2D3 or (iii) an immobilized fragment of suPAR or D2D3 of suPAR, wherein competitive inhibition of at least about 20%, preferably 50%, more preferably 70% and most preferably 90%, indicates that an antibody or ligand binds to the same epitope.

In its preferred embodiments, this invention is directed to a radioconjugate comprising a chelating linker that binds to a radioactive metal and links the radioactive metal to a mAb specific for human urokinase plasminogen activator receptor (uPAR). Also provided is a radioconjugate comprising a mAb specific for human uPAR to which is linked a radioactive metal. Preferably, the mAb is MNPR-101.

The radioactive metal is preferably linked to the mAb through a chelating linker, such as diethylenetriamine pentaacetate (DTPA) or deferoxamine (DFO), dodecane tetraacetic acid (DOTA) and Macropa-NCS (CAS No.: 2146095-31-8)

The radioactive metal is preferably an alpha particle emitter or alpha particle generator, such as ²¹²Pb, ²¹¹At or ²²⁵Ac.

The above radioconjugate preferably is one that binds to the surface of immune cells, preferably of the myeloid lineage, that express uPAR. Preferred myeloid cells are macrophages, monocytes and/or neutrophils that are hyperstimulated. The binding of the radioconjugate results in diminution of the number or activity of said cells, such as by eradication or destruction of these cells.

Also provided is a pharmaceutical composition comprising
(a) the above radioconjugate; and
(b) a pharmaceutically acceptable carrier or excipient, preferably one suitable for injection or oral administration.

The present invention is also directed to a method for treating or ameliorating the symptoms of severe respiratory distress in a subject in need thereof, comprising administration of the a effective amount of the radioconjugate or pharmaceutical composition. Examples of respiratory distress for which this method is applicable is resulting from bacterial sepsis, or infection by a respiratory virus, preferably the SARS-Cov2 virus. In the above methods the subject is preferably a human.

Also provide is the use of the above radioconjugate or pharmaceutical composition for treating or ameliorating the symptoms of severe respiratory disease in a subject, wherein an effective amount of the radioconjugate or composition is administered to a subject with the severe respiratory distress. In another embodiment, this invention provides use of a radioconjugate as above for the manufacture of a medicament for treatment of severe respiratory distress in a subject in need thereof.

### BRIEF DESCRIPTION OF THE DRAWINGS

FIG. 1 is a flow diagram depicting the inflammatory response to virus infection (as published in Channappanavar and Perlman. Sem Immunopathol 2017; 39:529-39).

FIG. 2 shows that biotinylated MNPR-101 binds saturably to suPAR.

FIG. 3 shows the binding of MNPR-101 to Monocytes and Neutrophils.

FIG. 4 shows a comparison of uPAR binding activity of MNPR-101, MNPR-101-DTPA alone (1.5 ratio) and MNPR-101-DTPA+Indium.

FIG. 5 shows a comparison of uPAR binding activity of MNPR-101, MNPR-101-DFO conjugate alone (1.9 ratio) and MNPR-101-DFO+Zr.

### DETAILED DESCRIPTION

One of the present inventors and colleagues found earlier that mAbs that target the uPA/uPAR complex or the uPAR-integrin complex are useful in the treatment and/or diagnosis of cancer. To date, the present inventors believe that no antibodies have been described that recognize the uPA-uPAR complex but not (a) uPAR or uPA individually or (b) uPAR in the presence of uPA (i.e., ligand occupied uPAR).

Further, the uPA-uPAR complex or uPAR alone have other “downstream” ligands such as integrins, low-density lipoprotein receptor-related protein (LRP) and other binding partners. These downstream interactions are believed to be important to the processes of cell migration, invasion and proliferation. It is thus desirable processes to target these processes therapeutically or detect the process or their interacting components diagnostically.

In addition to specific antibodies that target these interactions, as described in more detail below, this invention is also directed to methods for detecting antibodies that bind exclusively to the uPA-uPAR complex or that inhibit downstream interactions of uPAR.

The Antibody Approach

The present inventors have generated a panel of mAbs targeting uPAR. uPAR is an ideal target for antibodies because it is expressed on the cell surface. Expression of uPAR at the tumor-vasculature interface (on invasive tumor cells, angiogenic endothelial cells, or tumor-associated macrophages) as well as on hyperstimulated myeloid inflammatory cells involved in the pathogenesis of severe COVID-19 disease, suggests that antibodies targeting this protein would not suffer the same barriers to diffusion that have led to the failure of other mAbs to enter tumors or tissues and serve as diagnostic agents or exert therapeutic effects. Importantly, uPAR is not normally expressed on quiescent tissues, which should minimize the potential for toxicity when employing a therapeutic Ab and minimize non-specific signals (or false positives) when employing a diagnostic Ab.

The present invention is directed to uPAR-targeted precision radioimmunotherapeutic (uPRIT) that represents a novel approach to the treatment of severe respiratory distress, such as COVID-19 disease. uPAR is expressed by activated macrophages and neutrophils in COVID-19 patients that have or are at high risk of developing SARS based on several lines of evidence. The expression of uPAR is a hallmark of the alternative activation of myeloid cells that cause hyperinflammatory uPAR is found on activated macrophages, neutrophils and fibroblasts in acute lung injury and other organ injury in models of ARDS but not in normal quiescent adult tissue (Mazar et al., supra; Marudamuthu et al., supra).

A soluble form of uPAR (suPAR) that circulates and can be measured in plasma is shed from the surface of neutrophils and macrophages in ARDS and correlates with the increased hyperimmune response mediated by these cells (Gussen H supra; Liu G et al., supra. Ni W et al., supra). suPAR levels correlate with the severity, inflammation and mortality in patients that developed ARDS and are prognostic for the development of ARDS in patients with sepsis (Chen D et al., supra). suPAR is also prognostic for ICU mortality in ARDS patients (Geboers D G et al., supra) and a marker of infection in patients that develop acute kidney injury from the infection Hall A et al., supra) Finally, a recently published study demonstrates that suPAR is an early predictor of severe respiratory failure (BARS) in COVID-19 (Rovina N et al., supra). In this study, an increase in neutrophils also correlates with the development of COVID-19 respiratory distress. Targeting uPAR could lead to the rapid depletion of activated myeloid cells and would attenuate the cytokine storm and its sequelae (ARDS, coagulopathy, decreased T cells). This would allow the host immune system to restore normal function as described by Diao el al. (supra), in COVID-19 patients that recover from SARS/ARDS and allow healing from the infection.

uPRIT is designed to deliver low dose radioactivity precisely only to the alternatively activated myeloid cells associated with severe COVID-19 while sparing normal tissues. By reducing or eliminating these cells, the cytokine storm that leads to SARS/ARDS and systemic hyperinflammation will be reduced.

Rather than targeting individual cytokines produced by macrophages and neutrophils, the goal of most current clinical trials, uPRIT is designed to eradicate the source of these cytokines to mitigate cytokine storms.

A number of recently reported and planned studies support this approach. Individual cytokine targeted therapies (anti-TNF, anti-IL-6 and anti-IL-1β) are exhibiting hints of efficacy in the trials currently underway. The IL-6 blocking mAb immunotherapy, tociluzumab, anecdotally demonstrates a trend toward lower mortality and less severe respiratory systems as reported in several case reports and in small, single arm trials or retrospective analyses (Campochiaro C of al., Eur J Intern Med. 2020; 76:43-9; Wang L et al., Eur Rev Med Pharmacol Sci. 2020, 24:5783-5787; Borku Uysal B et al., J Med Virol. 2020 Jun. 2). However, despite hints of clinical benefit, patients receiving tocilizumab still develop severe COVID-19, SARS/ARDS and go on to die, suggesting that targeting a single cytokine such as IL-6 may be beneficial but not sufficient to completely mitigate severe COVID-19 mortality and morbidity.

Several recent studies have recently opened to test an antibody (TJ003234) that inhibits GM-CSF, a growth factor that stimulates the production of monocytes/macrophages and neutrophils in the bone marrow, or an antibody against the GM-CSF receptor (mavrilumab). These studies support the multi-cytokine approach since reduction in the production of monocyte/macrophages and neutrophils would also reduce the expression of multiple cytokines (NCT04341116. Study of TJ003234 (Anti-GM-CSF Monoclonal Antibody) in Subjects With Severe Coronavirus Disease 2019; NCT04397497. Mavrilimumab in Severe COVID-19 Pneumonia and Hyper-inflammation (COMBAT-19).

However, the targeting monocyte/macrophages and neutrophils should preferably be focused on those that are alternatively activated rather than on all monocyte/macrophages and neutrophils, (what blocking GM-CSF would achieve). Restoration of normal immune function will require the presence of normal myeloid cells so drawbacks of targeting GM-CSF or its receptor are the undesired depletion of all monocyte/macrophages and neutrophils that would be expected to persist for a long time when an antibody therapeutic is used.

The present inventors have developed a murine and a humanized mAb, MNPR-101, that directly binds to uPAR with high affinity and specificity (see, for example, Mazar et al., supra; and U.S. Pat. No. 8,101,726).

According to the present invention MNPR-101 (or another uPAR specific mAb such as MNPR-102) serves as a scaffold to design a uPAR-specific PRIT (“uPRIT”) that will deliver a therapeutic radionuclide, preferably an α-particle emitting radionuclide, only to the alternatively activated myeloid immune cells expressing uPAR while sparing normal cells and tissue that do not express uPAR or express very low levels. The uPRIT has been designed with the following operating characteristics:
Selective targeting and eradication of uPAR-expressing alternatively activated myeloid immune cells
Sparing of normal tissues including, in particular, non-activated immune cells and bone marrow
Judicious choice of alpha emitter to deliver a low dose of radiation only to the desired target cells which will internalize of uPRIT through cell surface uPAR
The alpha emitter is only cytotoxic once it is inside the cell, preventing bystander effects to neighboring cells when it is still on the cell surface
Choice of an alpha emitter with a short half-life that so that disarms uPRIT that is not taken up by the target cells (alternatively activated myeloid cells is disarmed, thereby minimizing any systemic or off-target effects

There is extensive rationale for using low dose radiation therapy (LD-RT) to treat hyperimmune responses and hyperinflammation in the lung (Montero A et al., Clin Transl Oncol. 2020 May 25:1-4). LD-RT has been used for many years to treat hyperinflammation in pneumonia; several recent studies using LD-RT to treat SARS/ARDS in COVID-19 patients have been proposed and are in the process of opening at various sites around the world (trial identifiers: NCT04377477, NCT04366791, NCT04414293). All of these studies are limited by the use of external beam radiation, which lacks the precision of the present targeted approach. Even though LD-RT generally uses a low dose of radiation (1 Gy or less), it is impossible to deliver this dose selectively and only to the hyperimmune cells; hence there is always radiation spillover to normal tissues. Further, in severe COVID-19, the hyperinflammation associated with a cytokine storm spreads beyond the lung and may affect kidney, brain and heart, rendering external beam radiation impractical with severe adverse events. The uPRIT approach of the present invention allows for precise and simultaneous targeting only to the cells of interest in multiple organ sites.

The present approach begins with the generation of a series of lead radiotherapeutic conjugates based on the MNPR-101 scaffold, their testing for in vitro uptake by and cytotoxicity to uPAR-expressing myeloid cells, test for selectivity against non-activated (uPAR null) and alternatively activated (uPAR positive) myeloid cells, and evaluation of the best uPRIT candidates in animal models of efficacy and toxicity with the goal of advancing the best uPRIT.

Extensive data from the literature as well as the studies of the present inventors and their colleagues documenting the highly selective expression of uPAR in inflammatory cells of the myeloid lineage but rarely in healthy human adult tissue. This supports the safety of such an approach. In addition to supportive histology and cell line data, several phase 1 PET imaging studies using a peptide that binds to uPAR in patients with advanced solid cancer have confirmed that uPAR is not expressed in most healthy tissues (Persson M et al., Theranostics, 2015, 5:1303-16; Persson M and Kjaer A, Clin Physiol Funct Imaging. 2013, 33:329-37; Skovgaard D et al., PET Clin. 2017, 12:311-19).

MNPR-101 (previously known as huATN-658) is a humanized (96% human sequence) mAb developed against human uPAR. MNPR-101 targets a previously unidentified epitope in uPAR, which has been demonstrated to mimic the CD11b binding site on uPAR (Xu X et al., PLoS One. 2014, 9: e85349). CD11b expression is a marker of myeloid immune cell activation (Pinsky M R, Contrib Nephrol. 2001, 132:354-66), i.e., that of macrophages and neutrophils and is involved in myeloid cell adhesion and infiltration when it interacts with uPAR on these cells (Gu J M et al., J Cell Physiol. 2005, 204:73-82). The present inventors and colleagues have also completed studies in collaboration with the NCI NExT program that developed methods for conjugating MNPR-101 with chelators without altering its binding activity, providing a template from which to develop a uPRIT. These chelators, conjugated to MNPR-101 or other mAbs are used herein to bind to and deliver alpha emitters or imaging metals to uPAR-expressing cells

A Master Cell Bank (MCB) that produces MNPR-101 has been manufactured and released under cGMP and upstream and downstream process development and analytical methods for manufacturing and release of MNPR-101 have been developed. Manufacturing to date has been scaled up to a 500 L bioreactor and an engineering run has been completed demonstrating proof of process. At the 500 L scale, MNPR-101 is manufactured that is >99% pure and provides sufficient scaffold material to make enough uPRIT to treat several hundred patients in early clinical studies.

In the present invention, a preferred embodiment is to use the MNPR-101 mAb that is combined with an alpha emitter such as ²¹²Pb. ²¹²Pb has a 10.65 hr half-life and is readily available from generators based on the parent of ²¹²Pb which is ²²⁴Ra. The half-life of ²¹²Pb makes it amenable to dose preparation in regional locations and shipment overnight to patient locations for administration by trained medical professionals. The use of a generator system allows for repeated elution of ²¹²Pb activities for multiple doses prepared for shipment. The use of an alpha emitting radionuclide for a therapeutic purpose has previously been approved by the FDA (Xofigo®).

As clinical development candidates are identified, a “cold kit” would be generated that comprises of MNPR-101 conjugated to a chelator. These cold kits are provided to hospitals as sterile solutions that would be loaded with the radionuclide to generate uPRIT at the treating hospital just prior to administration to a severe COVID-19 patient. This program is currently at a TRL 5 of technical maturity since the key elements for preparing a uPRIT for clinical use have been demonstrated.

Lead Screening and Optimization

uPRITs based on the MNPR-101 humanized antibody backbone using different chelators are made and tested. Initial focus is on ²¹²Pb in view of its decay rate and half-life characteristics. ²¹²Pb has a half-life of about 11 hours; while not an alpha emitter on its own, it decays to ²¹²Bi which is an alpha emitter with a ¹ hour half-life that quickly becomes inert after several half-lives). The use of ²¹²Pb (actually its precursor ²²⁴Ra, which decays to ²¹²Pb and allows this radioisotope to be prepared and shipped for use overnight) provides a radioisotope with characteristics amenable to preparation, shipment and delivery to clinical site, administration, and rapid decay to disarm the uPRIT.

Examples of preferred embodiments are:
²¹²Pb-MACROPA-MNPR-101 (Macropa-NCS—CAS No.: 2146095-31-8)
²¹²Pb-DOTA-MNPR-101. Dodecane tetraacetic acid (DOTA (also known as tetraxetan) is preferred for proof of concept since it is in the public domain and a MNPR-101 conjugate has already been generated and shown to retain its uPAR binding activity (FIG. 3).
Site-specific conjugations as described below may modify the half-life of the antibody and allow longer half-life radioisotopes.
Other alpha emitting isotopes that can be used as ²¹²Pb back-ups include: ²¹¹At (half-life 7.2 hr.) or ²²⁵Ac (half-life 9.9 days) as examples.

Random conjugation of radionuclide chelators at stoichiometries of ≤2:1 retain full or near full binding activity to uPAR. Site-specific conjugation without introduction of modifications into the MNPR-101 antibody is attained using an enzymatic process described by S. Jeger et al. (Angew Chem Int Ed Engl. 2010; 49: 9995-10010) and P. Dennler et al., Bioconjug Chem. 2014; 25:569-78). The process results in antibodies conjugated with chelator at two specific sites. The Ab is deglycosylated by the enzyme N-glycosidase F (PNGase F) and subsequently incubated with the enzyme microbial transglutaminase (MTGase). MTGase creates a covalent bond between the chelator and the amino acid residue glutamine. There are only two possible glutamines for this reaction in the antibody, because the enzyme only recognizes glutamine flanked by specific amino acids in a flexible region of the antibody. These specific glutamines are located in the Fc region, one at each heavy chain. Conjugation of the antibody in this way also decreases the plasma half-life substantially providing more flexibility for the choice of radioisotope (Strop P et al., Chem Biol. 2013, 20:161-7).

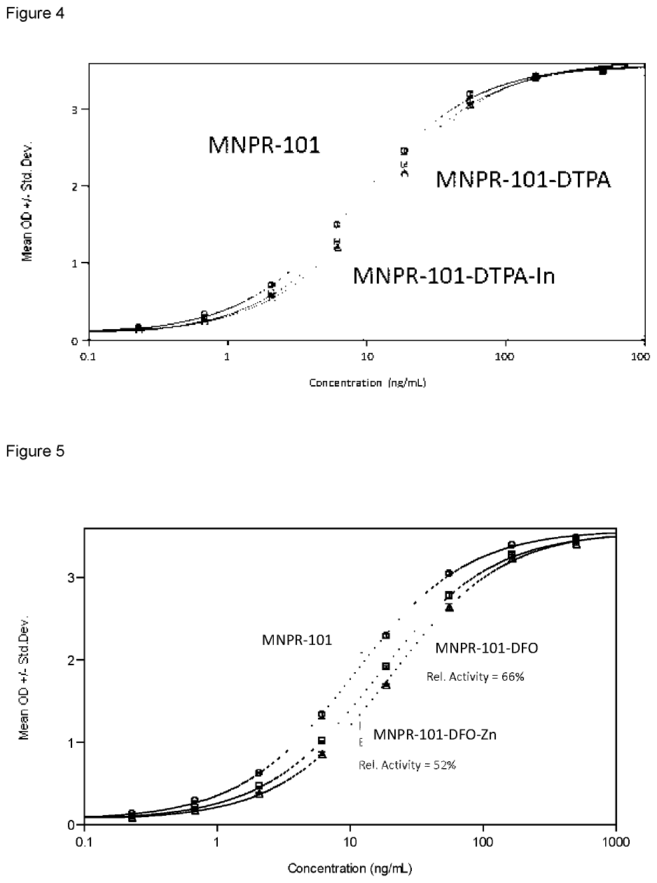

The conjugation of a number of different chelators and conjugation chemistries in addition to those shown (FIGS. 3 and 4) and described above are within the scope of this invention. See, for example, Tafreshi N K, et al. Development of Targeted Alpha Particle Therapy for Solid Tumors. Molecules. 2019 November; 24:4314; and Fay R and Holland J P. The Impact of Emerging Bioconjugation Chemistries on Radiopharmaceuticals. J Nucl Med. 2019 May; 60:587-91, which are incorporated by reference.

Random and site-specific conjugations at different stoichiometries are compared using BIAcor, solid phase and cell-based binding assays. A validated uPAR solid phase binding assay was previously developed for release testing of MNPR-101 as described in FIG. 4. The present inventor (Mazar) has extensive expertise with both BIAcor and whole cell binding assays (for example, to monocyte cell lines or to freshly isolated monocytes differentiated to macrophages). Binding assays compare the affinities of MNPR-101, MNPR-101 plus chelator, and MNPR-101 plus chelator loaded with the Pb isotope.

The present inventors collaborate with IsoTherapeutics Group, LLC in Angleton, TX to synthesize and test radioimmunoconjugates based on the MNPR-101 scaffold. IsoTherapeutics Group is a Contract Research Organization with expertise in radioconjugate discovery, development and GMP manufacturing. The present inventors will generate 3-5 radioconjugates that retain all or most of the binding activity of the unmodified anti-uPAR mAb. These will be stratified based on in vitro binding activity. Antibodies against mouse uPAR will be made and evaluated in a similar way for use as surrogates for the human uPAR in some of the rodent models due to species-specificity of binding to uPAR.

Proof of Concept: In Vitro and In Vivo Studies

Macrophage and neutrophil immortalized cell lines are used to assess cytotoxic potential of ²¹²P-MNPR-101 conjugates. In initial screens, suspensions of cultured cell cultures of U937, HL-60 and THP-1 lines will establish dose-response of the preferred conjugates. Also, induction of differentiation of HL-60 cells upregulates uPAR expression is used to compare cytotoxicity in wild type (uPAR null) and differentiated (uPAR-positive) HL-60. These cell are co-cultured with non-uPAR expressing endothelial cells to show specificity of cytotoxic effects in only uPAR cells. Finally, bronchoalveolar lavage fluids (BAL) from patients with severe COVID-19, which are rich in macrophages and neutrophils, are tested with the uPRIT. Control BAL are from patients with other conditions (non-SARS-CoV2).

In vivo studies rely on numerous rodent and rabbit models of ARDS induced by a variety of pathogens including viruses and bacterial components that recapitulate human ARDS and include inflammatory cell response and systemic sequelae of cytokine release syndrome. TLI also uses models of sepsis and induced cytokine release syndrome. Contemporary models of cytokine release syndrome involve transplant of human bone marrow into bone marrow depleted mice (either by genetic means or irradiation) and recapitulate the myeloid cell-dominant progression of cytokine release syndrome seen in poor prognosis SARS-CoV2. Candidate agents are also evaluated in a volumetric rabbit model or ARDS, in which lung damage is induced by ventilator tidal volumes.

The present inventors will carry out animal studies possibly in collaboration with virologists at the Texas Biomedical Research Institute in San Antonio, who run SARS-CoV2 models in rodents and particularly in baboons because this group has adequate biocontainment facilities in addition to expertise in models of respiratory disease and distress.

Variable (V) Region Amino Acid Sequences of two Preferred mAbs

mAb MNPR 101 (Formerly ATN-658): Variable Region Sequences

The consensus amino acid sequence (single-letter code) of the light chain variable region (V_{L}) and heavy chain variable region (V_{H}) polypeptides of mAb ATN-658 are shown below. cDNA was prepared from total RNA extracted from the hybridoma expressing ATN-658 and the variable regions were cloned, amplified and sequenced using standard techniques. The complementarity-determining regions (CDRs) for each variable region are highlighted

[symbol]

[symbol]

| MNPR-101 V_{L} Consensus Protein (SEQ ID NO: 1): | |
|---|---|
| 1 | DIXLTQSPLT LSVTIGQPAS ISC [symbol] W LLQRPGQSPK |
| 51 | RLIY [symbol] GVPDRFTGS GSGTDFTLKI SRVEAEDLGV YYC [symbol] |
| 101 | LTFGAGTKLE LKL |
| MNPR-101 V_{H} Consensus Protein (SEQ ID NO: 2) | |
| 1 | VQLQESGPEL VKTGASVKIS CKAS [symbol] WVKQSH GKSLEWIG [symbol] |
| 51 | [symbol] RATFT VDTSSRTAYM QFNSLTSEDS AVYYCAR [symbol] |
| 101 | [symbol] WGQ GTTVTVS |

TABLE 2
Characteristics of CDRs of MNPR-101 L and H Chains

| | | | SEQ |
|---|---|---|---|
| | No. of | | ID |
| CDR* | residues | Sequence¹ | NO: |
| CDR L1 | 16 | KSSQSLLDSDGKTYLN | 3 |
| CDR L2 | 7 | LVSKLDS | 4 |
| CDR L3 | 9 | WQGTHFPLT | 5 |
| CDR H1 | 10 | GYSFTSYYMH | 6 |
| CDR H2 | 17 | EINPYNGGASYNQKIKG | 7 |
| CDR H3 | 10 | SIYGHSVLDY | 8 |

*CDR-L1: first CDR of L chain; CDR-H2: 2^{nd} CDR of H chain, etc.

mAb MNPR-102: Variable Region Sequences

Amino acid sequence (single-letter code) of the light chain (V_{L}) and heavy chain (V_{H}) variable regions of monoclonal antibody MNPR-102. cDNA was prepared from total RNA extracted from the hybridoma expressing MNPR-102 and the variable regions cloned, amplified and sequenced using standard techniques. The complementarity-determining regions (CDRs) for each variable region are highlighted in red.

### MNPR-102 V_{L} Consensus Protein Sequence (SEQ ID NO:9)

| 1 | DIVLTQSPDI TAASLGQKVT ITC [symbol] WYQQKSG TSPKPWIF [symbol] |
|---|---|
| 51 | [symbol] GVPAR FSGSGSGTSY SLTISSMEAE DAAIYYC [symbol] FGGGT |
| 101 | KLEIKR |
| MNPR-102 V_{H} Consensus Protein Sequence (SEQ ID NO: 10) | |
| 1 | VKLQQSGPEV VKPGASVKIS CKAS [symbol] WVKQRP GQGLEWIG [symbol] |
| 51 | [symbol] KATLT ADTSSSTAYM QLSSLTSEDS AVYFCAR [symbol] |
| 101 | [symbol] WGQG TTVTVSS |

TABLE 3
Characteristics of the CDRs of MNPR-102

| | No. of | | SEQ |
|---|---|---|---|
| CDR* | residues | Sequence | ID NO: |
| CDR L1 | 10 | SASSSVSYMH | 11 |
| CDR L2 | 7 | EISKLAS | 12 |
| CDR L3 | 8 | QQWNYPFT | 13 |
| CDR H1 | 10 | GYSFTNFYIH | 14 |
| CDR H2 | 17 | WIFHGSDNTEYNEKFKD | 15 |
| CDR H3 | 9 | WGPHWYFDV | 16 |

*CDR-L1: first CDR of L chain; CDR-H2: 2^{nd} CDR of H chain,
etc.

According to the present invention, an Ab or mAb, has “essentially the same antigen-binding characteristics” as a reference mAb if it demonstrates a similar specificity profile (e.g., by rank order comparison), and has affinity for the relevant antigen (e.g., uPA-uPAR complex) within 1.5 orders of magnitude, more preferably within one order of magnitude, of the reference Ab.

The antibodies and conjugates can be evaluated for direct anti-angiogenic activity in an in vivo Matrigel plug model. Radioiodinated antibodies and conjugates are used to test Ab internalization using, for example, MDA MB 231 cells which express both receptor and ligand. Antibody internalization is also measured in the presence of PAI-1:uPA complexes.

TABLE 4
uPAR epitope sequences

| | Amino acids of | SEQ ID |
|---|---|---|
| Sequence | Human uPAR1 | NO: |
| CCTKSGCNHPDLDVQYPSG | 265-283 | 17 |
| CCTKSGCNHPDLDVQYPS | 265-282 | 18 |
| CCTKSGCNHPDLDVQYP | 265-281 | 19 |
| CCTKSGCNHPDLDVQY | 265-280 | 20 |
| CCTKSGCNHPDLDVQ | 265-279 | 21 |
| CGSSDMSCERGRHQSL | 98-114 | 22 |
| KSGCNHPDLD | 268-277 | 23 |

¹The amino acid numbering reflects the processed form of uPAR

### Chimeric/Humanized Antibodies

The chimeric antibodies of the invention comprise individual chimeric H and L Ig chains. The chimeric H chain comprises an antigen binding region derived from the H chain of a non-human Ab specific for e.g., uPA/uPAR or uPAR-integrin complex, for example, MNPR-101 or mAb MNPR-102, which is linked to at least a portion of a human CH region. A chimeric L chain comprises an antigen binding region derived from the L chain of a non-human Ab specific for the target antigen, such as the hybridoma MNPR-101 or MNPR-102r, linked to at least a portion of a human CL region. As used herein, the term “antigen binding region” refers to that portion of an Ab molecule which contains the amino acid residues that interact with an antigen and confer on the Ab its specificity and affinity for the antigen. The Ab region includes the “framework” amino acid residues necessary to maintain the proper conformation of the antigen-binding (or “contact”) residues.

As used herein, the term “chimeric antibody” includes monovalent, divalent or polyvalent Igs. A monovalent chimeric Ab is an HL dimer formed by a chimeric H chain associated through disulfide bridges with a chimeric L chain. A divalent chimeric Ab is tetramer H₂L₂ formed by two HL dimers associated through at least one disulfide bridge. A polyvalent chimeric Ab can also be produced, for example, by employing a CH region that aggregates (e.g., from an IgM H chain, termed the μ chain).

The invention also provides for “derivatives” of the mouse mAbs or the chimeric Abs, which term includes those proteins encoded by truncated or modified genes to yield molecular species functionally resembling the Ig fragments. The modifications include, but are not limited to, addition of genetic sequences coding for cytotoxic proteins such as plant and bacterial toxins. The fragments and derivatives can be produced from any of the hosts of this invention.

Antibodies, fragments or derivatives having chimeric H chains and L chains of the same or different V region binding specificity, can be prepared by appropriate association of the individual polypeptide chains, as taught, for example by Sears et al., Proc. Natl. Acad. Sci. USA 72:353-357 (1975). With this approach, hosts expressing chimeric H chains (or their derivatives) are separately cultured from hosts expressing chimeric L chains (or their derivatives), and the Ig chains are separately recovered and then associated. Alternatively, the hosts can be co-cultured and the chains allowed to associate spontaneously in the culture medium, followed by recovery of the assembled Ig, fragment or derivative.

The antigen binding region of the chimeric Ab (or a human mAb) of the present invention is derived preferably from a non-human Ab specific for e.g., uPA/uPAR or uPAR-integrin complex. Preferred sources for the DNA encoding such a non-human Ab include cell lines which produce Ab, preferably hybridomas. Preferred hybridomas are the MNPR-101 hybridoma cell line (ATCC Accession No. PTA-8191 and MNPR-102 (ATCC Accession No. PTA 8192) which were produced as described above and whose V regions have the sequences shown above.

Thus, a preferred chimeric Ab (or human Ab) has a V_{L} sequence SEQ ID NO:1 and a V_{H} sequence SEQ ID NO:2 which are the consensus sequences of mAb MNPR-101. The residues of these V regions that are not in the CDR regions may be varied, preferably as conservative substitutions, as long as the V region results in an Ab with the same antigen-specificity and substantially the same antigen-binding affinity or avidity, preferably at least 20% of the affinity or avidity of an Ab wherein the V_{L} sequence is SEQ ID NO:1 and the V_{H} sequence is SEQ ID NO:2. It is preferred that in this chimeric (or human) Ab, the three CDR regions of the V_{L} chain are SEQ ID NO:3, 4 and 5 and the three CDR regions of the V_{H} chain are SEQ ID NO:6, 7 and 8.

Another preferred chimeric Ab (or human Ab) has a V_{L} sequence SEQ ID NO:9 and a V_{H} sequence SEQ ID NO:10 which are the consensus sequences of mAb MNPR-102. The residues of these V regions that are not in the CDR regions may be varied, preferably as conservative substitutions, as long as the V region results in an Ab with the same antigen-specificity and substantially the same antigen-binding affinity or avidity, preferably at least 20% of the affinity or avidity of an Ab wherein the V_{L} sequence is SEQ ID NO:9 and the V_{H} sequence is SEQ ID NO:10. It is preferred that in this chimeric Ab, the three CDR regions of the V_{L} chain are SEQ ID NO:11, 12 and 13 and the three CDR regions of the V_{H} chain are SEQ ID NO:14, 15 and 16.

Preferred nucleic acid molecules for use in constructing a chimeric Ab (or human Ab) of this invention are (a) a nucleic acid molecule with a coding sequence that encodes a V_{L} region with the sequence SEQ ID NO:1 and (b) a nucleic acid molecule with a coding sequence that encodes a V_{H} chain with the sequence SEQ ID NO:2. Also preferred is a nucleic acid molecule that encodes a V_{L} region comprising the three CDRs SEQ ID NO:3, 4 and 5 and a nucleic acid molecule that encodes a V_{H} region comprising the three CDRs SEQ ID NO:6, 7 and 8.

Another set of preferred nucleic acid molecules for use in constructing a chimeric Ab (or human Ab) of this invention are (a) a nucleic acid molecule with a coding sequence that encodes a V_{L} region with the sequence SEQ ID NO:9 and (b) a nucleic acid molecule with a coding sequence that encodes a V_{H} chain with the sequence SEQ ID NO:10. Also preferred is a nucleic acid molecule that encodes a V_{L} region comprising the three CDRs SEQ ID NO:11, 12 and 13 and a nucleic acid molecule that encodes a V_{H} region comprising the three CDRs SEQ ID NO:14, 15 and 16.

Alternatively, the non-human Ab producing cell from which the V region of the Ab of the invention is derived may be a B lymphocyte obtained from the blood, spleen, lymph nodes or other tissue of an animal immunized with D2D3 of suPAR. The Ab-producing cell contributing the nucleotide sequences encoding the antigen-binding region of the chimeric Ab of the present invention may also be produced by transformation of a non-human, such as a primate, or a human cell. For example, a B lymphocyte which produces an Ab specific, e.g., uPA/uPAR or uPAR-integrin complex may be infected and transformed with a virus such as Epstein-Barr virus to yield an immortal Ab producing cell (Kozbor et al. Immunol. Today 4:72-79 (1983)). Alternatively, the B lymphocyte may be transformed by providing a transforming gene or transforming gene product, as is well-known in the art. Preferably, the antigen binding region will be of murine origin. In other embodiments, the antigen binding region may be derived from other animal species, in particular rodents such as rat or hamster.

The murine or chimeric mAb of the present invention may be produced in large quantities by injecting hybridoma or transfectoma cells secreting the Ab into the peritoneal cavity of mice and, after appropriate time, harvesting the ascites fluid which contains a high titer of the mAb, and isolating the mAb therefrom. For such in vivo production of the mAb with a non-murine hybridoma (e.g., rat or human), hybridoma cells are preferably grown in irradiated or athymic nude mice.

Alternatively, the antibodies may be produced by culturing hybridoma (or transfectoma) cells in vitro and isolating secreted mAb from the cell culture medium.

Human genes which encode the constant C regions of the chimeric antibodies of the present invention may be derived from a human fetal liver library or from any human cell including those which express and produce human Igs. The human CH region can be derived from any of the known classes or isotypes of human H chains, including γ, μ, α, δ or ε, and subtypes thereof, such as G1, G2, G3 and G4. Since the H chain isotype is responsible for the various effector functions of an Ab, the choice of CH region will be guided by the desired effector functions, such as complement fixation, or activity in Ab-dependent cellular cytotoxicity (ADCC). Preferably, the CH region is derived from γ1 (IgG1), γ3 (IgG3), γ4 (IgG4), or μ (IgM).

The human CL region can be derived from either human L chain isotype, κ or λ.

Genes encoding human Ig C regions are obtained from human cells by standard cloning techniques (Sambrook, J. et al., Molecular Cloning: A Laboratory Manual, 2nd Edition, Cold Spring Harbor Press, Cold Spring Harbor, NY (1989)). Human C region genes are readily available from known clones containing genes representing the two classes of L chains, the five classes of H chains and subclasses thereof. Chimeric Ab fragments, such as F(ab′)₂ and Fab, can be prepared by designing a chimeric H chain gene which is appropriately truncated. For example, a chimeric gene encoding an H chain portion of an F(ab′)₂ fragment would include DNA sequences encoding the CH₁ domain and hinge region of the H chain, followed by a translational stop codon to yield the truncated molecule.

Generally, the chimeric antibodies of the present invention are produced by cloning DNA segments encoding the H and L chain antigen-binding regions of a specific Ab of the invention, preferably non-human, and joining these DNA segments to DNA segments encoding human CH and CL regions, respectively, to produce chimeric Ig-encoding genes.

Thus, in a preferred embodiment, a fused gene is created which comprises a first DNA segment that encodes at least the antigen-binding region of non-human origin, such as a functionally rearranged V region with joining (J) segment, linked to a second DNA segment encoding at least a part of a human C region.

The DNA encoding the Ab-binding region may be genomic DNA or cDNA. A convenient alternative to the use of chromosomal gene fragments as the source of DNA encoding the murine V region antigen-binding segment is the use of cDNA for the construction of chimeric Ig genes, as reported by Liu et al. (Proc. Natl. Acad. Sci., USA 84:3439 (1987); J. Immunol. 139:3521 (1987), which references are hereby incorporated by reference. The use of cDNA requires that gene expression elements appropriate for the host cell be combined with the gene in order to achieve synthesis of the desired protein. The use of cDNA sequences is advantageous over genomic sequences (which contain introns), in that cDNA sequences can be expressed in bacteria or other hosts which lack appropriate RNA splicing systems.

### Pharmaceutical and Therapeutic Compositions and Their Administration

The compounds that may be employed in the pharmaceutical compositions of the invention include all of the polypeptide molecules, preferably Abs, described above, as well as the pharmaceutically acceptable salts of these compounds. Pharmaceutically acceptable acid addition salts of the compounds of the invention containing a basic group are formed where appropriate with strong or moderately strong, non-toxic, organic or inorganic acids by methods known to the art. Exemplary of the acid addition salts that are included in this invention are maleate, fumarate, lactate, oxalate, methanesulfonate, ethanesulfonate, benzenesulfonate, tartrate, citrate, hydrochloride, hydrobromide, sulfate, phosphate and nitrate salts.

Pharmaceutically acceptable base addition salts of compounds of the invention containing an acidic group are prepared by known methods from organic and inorganic bases and include, for example, nontoxic alkali metal and alkaline earth bases, such as calcium, sodium, potassium and ammonium hydroxide; and nontoxic organic bases such as triethylamine, butylamine, piperazine, and tri(hydroxymethyl)methylamine.

Therapeutically Conjugated Compositions

In a preferred embodiment, the mAbs describe herein are “therapeutically conjugated” and used to deliver a therapeutic radionuclide, preferably an alpha-emitting radionuclide, to the site to which the compounds home and bind, such as sites of tumor metastasis or foci of infection/inflammation, restenosis or fibrosis. The term “therapeutically conjugated” means that the modified mAb is conjugated to another therapeutic agent that is directed either to the underlying cause or to a “component” of inflammation or other pathology. A therapeutically conjugated mAb carries a suitable therapeutic moiety, which is preferably an alpha-emitting atom, in combination with a chelating/conjugating agent renders the mAb active in treating a target disease or condition. The therapeutic moiety may be bound directly or indirectly to the mAb. The therapeutically conjugated mAb is administered as pharmaceutical composition which comprises a pharmaceutically acceptable carrier or excipient, and is preferably in a form suitable for injection.

Examples of various therapeutic radionuclide useful herein, but not limited to, include ⁴⁷Sc, ⁶⁷Cu, ⁹⁰Y, ¹⁰⁹Pd, ¹²⁵I, ¹³¹I, ¹⁸⁶Re, ¹⁸⁸Re, ¹⁹⁹Au, ²¹¹At, ²¹²Pb, ²¹³Bi, ²²³Ra, ²²⁷Th, or ²²⁵Ac. These atoms can be conjugated to the peptide directly, indirectly as part of a chelate,

Preferred doses of the radionuclide conjugates are a function of the specific radioactivity to be delivered to the target site which varies with tissue, and vascularization, kinetics and biodistribution of the polypeptide “carrier,” the energy of radioactive emission by the nuclide, etc. Those skilled in the art of radiotherapy can adjust the dose of the antibody in conjunction with the dose of the particular nuclide to effect the desired therapeutic benefit.

A preferred embodiment will include a “cold kit” comprised of the mAb conjugated to a chelator that is combined with the “hot” kit, a pharmaceutically acceptable formulation of the radionuclide just prior to administration to a patient with severe COVID 19.

The compounds of the invention, as well as the pharmaceutically acceptable salts thereof, may be incorporated into convenient dosage forms, such as capsules, impregnated wafers, tablets or injectable preparations. Solid or liquid pharmaceutically acceptable carriers may be employed.

Solid carriers include starch, lactose, calcium sulfate dihydrate, terra alba, sucrose, talc, gelatin, agar, pectin, acacia, magnesium stearate and stearic acid. Liquid carriers include syrup, peanut oil, olive oil, saline, water, dextrose, glycerol and the like. Similarly, the carrier or diluent may include any prolonged release material, such as glyceryl monostearate or glyceryl distearate, alone or with a wax. When a liquid carrier is used, the preparation may be in the form of a syrup, elixir, emulsion, soft gelatin capsule, sterile injectable liquid (e.g., a solution), such as an ampoule, or an aqueous or nonaqueous liquid suspension. A summary of such pharmaceutical compositions may be found, for example, in Remington's Pharmaceutical Sciences, Mack Publishing Company, Easton Pennsylvania (Gennaro 18th ed. 1990).

The pharmaceutical preparations are made following conventional techniques of pharmaceutical chemistry involving such steps as mixing, granulating and compressing, when necessary for tablet forms, or mixing, filling and dissolving the ingredients, as appropriate, to give the desired products for oral, parenteral, topical, transdermal, intravaginal, intrapenile, intranasal, intrabronchial, intracranial, intraocular, intraaural and rectal administration. The pharmaceutical compositions may also contain minor amounts of nontoxic auxiliary substances such as wetting or emulsifying agents, pH buffering agents and so forth.

The present invention may be used in the diagnosis or treatment of any of a number of animal genera and species, and are equally applicable in the practice of human or veterinary medicine. Thus, the pharmaceutical compositions can be used to treat domestic and commercial animals, including birds and more preferably mammals, as well as humans.

The term “systemic administration” refers to administration of a composition or agent such as the polypeptide, described herein, in a manner that results in the introduction of the composition into the subject's circulatory system or otherwise permits its spread throughout the body, such as intravenous (i.v.) injection or infusion. “Regional” administration refers to administration into a specific, and somewhat more limited, anatomical space, such as intraperitoneal, intrathecal, subdural, or to a specific organ. Examples include intravaginal, intrapenile, intranasal, intrabronchial (or lung instillation), intracranial, intra-aural or intraocular. The term “local administration” refers to administration of a composition or drug into a limited, or circumscribed, anatomic space, subcutaneous (s.c.) injections, intramuscular (i.m.) injections. One of skill in the art would understand that local administration or regional administration often also result in entry of a composition into the circulatory system, i.e., so that s.c. or i.m. are also routes for systemic administration. Injectables or infusible preparations can be prepared in conventional forms, either as solutions or suspensions, solid forms suitable for solution or suspension in liquid prior to injection or infusion, or as emulsions. Though the preferred routes of administration are systemic, such as i.v., the pharmaceutical composition may be administered topically or transdermally, e.g., as an ointment, cream or gel; orally; rectally; e.g., as a suppository.

Other pharmaceutically acceptable carriers for polypeptide compositions of the present invention are liposomes, pharmaceutical compositions in which the active protein is contained either dispersed or variously present in corpuscles consisting of aqueous concentric layers adherent to lipidic layers. The active polypeptide is preferably present in the aqueous layer and in the lipidic layer, inside or outside, or, in any event, in the non-homogeneous system generally known as a liposomic suspension. The hydrophobic layer, or lipidic layer, generally, but not exclusively, comprises phospholipids such as lecithin and sphingomyelin, steroids such as cholesterol, more or less ionic surface active substances such as dicetylphosphate, stearylamine or phosphatidic acid, and/or other materials of a hydrophobic nature. Those skilled in the art will appreciate other suitable embodiments of the present liposomal formulations.

Therapeutic compositions may comprise, in addition to the uPRIT, one or more additional drugs, such as antiviral agents. In fact, pharmaceutical compositions comprising any known therapeutic in combination with the uPRITs disclosed herein are within the scope of this invention. The pharmaceutical composition may also comprise one or more other medicaments to treat additional symptoms for which the target patients are at risk, for example, anti-infectives.

The therapeutic dosage administered is an amount which is therapeutically effective, as is known to or readily ascertainable by those skilled in the art. The dose is also dependent upon the age, health, and weight of the recipient, kind of concurrent treatment(s), if any, the frequency of treatment, and the nature of the effect desired, such as, for example, anti-inflammatory effects or anti-bacterial effect.

Therapeutic Methods

The methods of this invention may be used to treat severe respiratory distress or ARDS in severe COVID-19 or other respiratory infections that lead to severe respiratory distress. A vertebrate subject, preferably a mammal, more preferably a human, is administered an amount of the compound effective to eliminate hyperactivated myeloid cells that are mediating the respiratory distress. The compound or pharmaceutically acceptable salt thereof is preferably administered in the form of a pharmaceutical composition as described above.

Doses of the uPRIT, preferably include pharmaceutical dosage units comprising an effective amount of the compound. Dosage unit form refers to physically discrete units suited as unitary dosages for a mammalian subject; each unit contains a predetermined quantity of active material calculated to produce the desired therapeutic effect, in association with the required pharmaceutical carrier. The specification for the dosage unit forms of the invention are dictated by and directly dependent on (a) the unique characteristics of the active material and the particular therapeutic effect to be achieved, and (b) the limitations inherent in the art of compounding such an active compound for the treatment of, and sensitivity of, individual subjects By an effective amount is meant an amount sufficient to achieve a steady state concentration in vivo which results in a measurable reduction in any relevant parameter of disease such as any accepted index of inflammatory reactivity, or a measurable prolongation of disease-free interval or of survival.

In one embodiment, an effective dose is preferably 10-fold and more preferably 100-fold higher than the 50% effective dose (ED₅₀) of the compound in an in vivo assay as described herein.

The amount of active compound to be administered depends on the precise peptide or derivative selected, the disease or condition, the route of administration, the health and weight of the recipient, the existence of other concurrent treatment, if any, the frequency of treatment, the nature of the effect desired, for example, inhibition of tumor metastasis, and the judgment of the skilled practitioner.

A preferred dose for treating a subject, preferably mammalian, more preferably human, is an amount of up to 20 mg of the uPRIT per kilogram of body weight. A typical single dosage of the antibody is between about 1 ng and about 50 mg/kg body weight. A total daily dosage in the range of about 0.1 milligrams to about 7 grams is preferred for intravenous administration. The foregoing ranges are, however, suggestive, as there are possible variables in an individual treatment regime.

An effective amount or dose of the uPRIT for inhibiting inflammatory myeloid cells in vitro is in the range of about 1 picogram to about 5 nanograms per cell. Effective doses and optimal dose ranges may be determined in vitro using the methods described herein.

A longer example of a disease or condition against which the above method is effective include fibrosis associated with a chronic inflammatory condition, such as lung fibrosis in COVID-19 disease.

Having now generally described the invention, the same will be more readily understood through reference to the following examples which are provided by way of illustration, and are not intended to be limiting of the present invention, unless specified.

### EXAMPLES

### Example 1—Analysis of Biotinylated MNPR-101

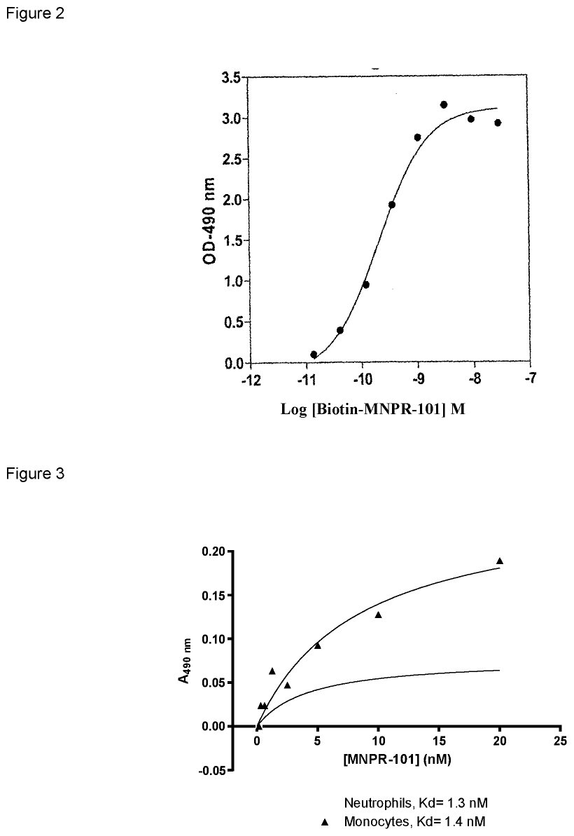

MNPR-101 (formerly huATN-658) was biotinylated using EZ-link™ sulfo-NHS-LC-biotin (Pierce Biotechnology Inc.) according to the manufactures instructions. Typically, a 20-fold molar excess of the biotin-labeling reagent was used to label MNPR-101 and unincorporated biotin removed from the labeled Ab using a size exclusion column. To ensure that the labeled Ab retained its affinity for uPAR, Biotin-MNPR-101 was tested in an ELISA assay for binding to suPAR. Bound Biotin-ATN-MNPR-101) was detected using horseradish peroxidase (HRP)-conjugated streptavidin. Biotin labeling did not reduce the affinity of the mAb for suPAR (FIG. 2). Similarly, biotin-MNPR-101 was also able to bind to whole cells that express uPAR in a saturable manner.

### Example 2—Binding of MNPR-101 to Monocytes and Neutrophils

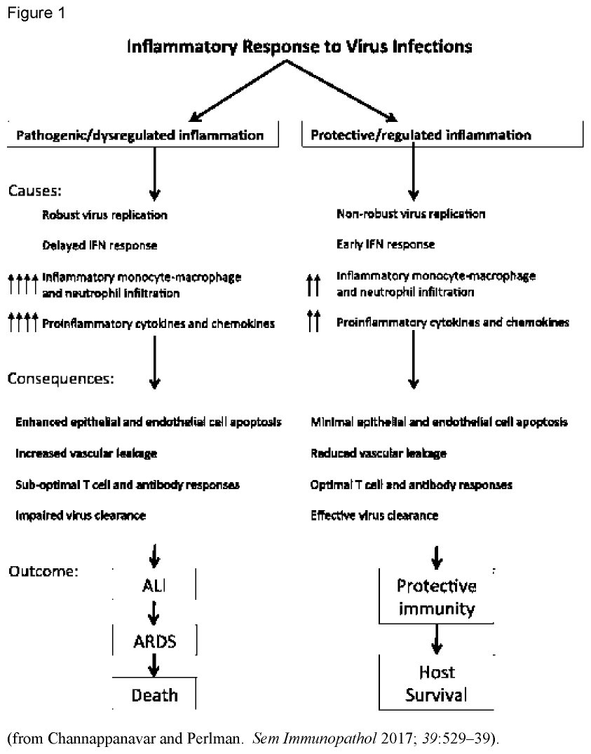

Whole blood was obtained from healthy human volunteers and PBMC was prepared using a Ficoll gradient. Neutrophils were purified by collecting the red blood cell (RBC)/granulocyte component followed by lysis of the RBC. PBMC were added to T75 flasks, incubated for 60 minutes and the unattached lymphocyte component was decanted, leaving monocytes loosely attached to the flask. Monocytes were removed by rapid wash and pipetting. The identity of neutrophils and monocytes was confirmed by flow cytometry using CD66b and CD14 antibodies, respectively. Neutrophils and monocytes were suspended in PBS in untreated polypropylene tubes and Biotinylated MNPR-101 at different concentrations was added to the tubes. After a 60 minute incubation, unbound biotin-MNPR-101 was removed by centrifuging cells, removing supernatants, washing with PBS, re-centrifuging and measuring bound biotin-MNPR-101 as described in FIG. 1. FIG. 3 shows that biotinylated MNPR-101 binds to both monocytes and neutrophils with a Kd in the nanomolar range.

### Example 3—Comparison of uPAR Binding Activity of MNPR-101 Constructs

Preparation and binding of MNPR-101-Diethylenetriamine pentaacetate (DTPA) Conjugates: SCN-CHX-A″-DTPA chelate (Macrocyclics Inc.) was dissolved in DMSO (20 mM). Approximately 12 equivalent of DTPA chelate was incubated with 5 mg of MNPR-101 in 1 mL saline for 5 hrs at 37° C. pH of the reaction was adjusted to 9 with 0.1 M Na₂CO₃. MNPR-101-DTPA conjugate was purified using PD10 column (GE Healthcare) with 0.9% NaCl. Antibody concentration was determined by the Lowry assay and number of chelates per antibody was determined by radioactive metal (⁵⁷Co) binding assay. Assay results showed that the yield of the isolated conjugate was ˜80% (3-4 mg) with <2 chelates per antibody molecule.

For In-labeling, ˜1-2 mg of this conjugate was incubated with 2 μL of 25 mM of InCl₃ solution in 50 mM NaOAc buffer (— 0.2 mL) for 1 hr at room temperature. Indium labeled uPAR conjugate was then purified using PD10 column with 0.9% NaCl. Concentration of labeled conjugate was determined by using the Lowry assay.

To verify the binding and specificity of binding activity of conjugated and/or labeled MNPR-101, a uPAR binding ELISA assay was performed. MNPR-101 or its conjugate was used as a test standard sample and a commercial human uPAR monoclonal antibody (mouse IgG, R & D Systems) was used as a positive control. Human IgG1 and mouse IgG1 were used as negative controls. The recombinant human soluble uPAR (suPAR) was used as the coating protein. HRP-conjugated goat anti-human antibody was used for detecting the presence of human antibody. HRP-conjugated goat anti-mouse antibody was used for detecting the presence of mouse antibody. The bound HRP antibodies were detected using the HRP reactive reagent TMB.

Two 96-well plates were coated with recombinant human suPAR (1 μg/mL) overnight. After removal of the coating reagent, the plate was washed 3 times with PBS-0.05% Tween20 (PBST), and blocked with PBS-1% BSA-5% sucrose for 1 h. After washing three times with PBS-0.05% Tween-20, MNPR-101 test or control samples were added in triplicate wells in 3 fold serial dilutions and incubated for 1.5 h and washed 3× in PBST. HRP goat anti-human IgG or goat anti-mouse IgG (diluted 1:10000 in PBS-1% BSA) was added (100 μL/well). After a 1 h incubation, plate wells were washed 3× in PBST and TMB substrate was added (50 μl/well). The reaction was arrested by adding 50 μl/well of 2N H₂SO₄ within 5 minutes. The plate was read at 450/570 nm. FIG. 4 shows that each of MNPR-101, MNPR-101-DTPA and MNPR-101-DPTA-In bind to soluble uPAR in a similar manner.

Next, uPAR binding activity of MNPR-101, MNPR-101-DFO conjugate alone (1.9 ratio) and MNPR-101-DFO+Zr was assessed.

Preparation and Binding of MNPR-101-Deferoxamine (DFO) Conjugates:

Approximately X equivalent (see below) of SCN-p-DFO chelate (Macrocyclics Inc.) in DMSO (20 mM) was incubated with 5 mg of MNPR-101 in 1 mL saline for 1 hr at 37° C. pH of the reaction was adjusted to 9 with 0.1 M Na₂CO₃. MNPR-101-DFO conjugate was then purified using PD10 column (GE Healthcare) with 0.9% NaCl. Antibody concentration was determined by Lowry assay and number of chelate per antibody was determined by radioactive metal (89Zr) binding assay. Assay results showed that the yield of the isolated conjugate was ˜80% (3-4 mg) with <2 chelates per antibody molecule.
1. X=4, Ratio: 1.9
2. X=3, Ratio: 1.4
3. X=2, Ratio: 0.5

For Zr-labeling, ˜1-2 mg of this conjugate was incubated with 2 μL of 25 mM of Zr oxalate solution in saline for 1 hr at room temperature. pH was adjusted to 8 using 2 M Na2CO3. Zirconium labeled uPAR conjugate was then purified using PD10 column with 0.9% NaCl. Concentration of labeled conjugate was determined by using the Lowry assay. Binding analysis was carried out as described for FIG. 4. The results are shown in FIG. 5.

All the references cited above are incorporated herein by reference in their entirety, whether specifically incorporated or not.

Having now fully described this invention, it will be appreciated by those skilled in the art that the same can be performed within a wide range of equivalent parameters, concentrations, and conditions without departing from the spirit and scope of the invention and without undue experimentation. In the event of any disagreement between the amino acid sequences disclosed above and those in the electronic or paper Sequence Listing filed later, the sequences above shall take precedence.

What is claimed is:

1. A radioconjugate comprising a chelating linker that binds to a radioactive metal and links the radioactive metal to a monoclonal antibody (mAb) specific for human urokinase plasminogen activator receptor (uPAR).

2. A radioconjugate comprising a monoclonal antibody (mAb) specific for human uPAR to which is linked a radioactive metal.

3. The radioconjugate of claim 1 or 2, wherein the mAb is MNPR-101.

4. The radioconjugate of claim 2 or 3, wherein the radioactive metal is linked to the mAb through a chelating linker.

5. The radioconjugate of any one of claims 1-4, wherein the linker is selected from the group consisting of diethylenetriamine pentaacetate (DTPA) or deferoxamine (DFO), dodecane tetraacetic acid (DOTA) and Macropa-NCS (CAS No.: 2146095-31-8).

6. The radioconjugate of any one of claims 1-5, wherein the radioactive metal is an alpha particle emitter or alpha particle generator.

7. The radioconjugate of claim 6, wherein the alpha particle emitter or generator is 212Pb, 211At or 225Ac.

8. The radioconjugate of any one of claims 1-7, that binds to the surface of immune cells that express uPAR.

9. The radioconjugate of claim 8, wherein the immune cells are cells of the myeloid lineage.

10. The radioconjugate of claim 8 or 9, wherein the immune cells are macrophages, monocytes and/or neutrophils.

11. The radioconjugate of any one of claims 8-10, wherein binding to uPAR results in diminution of the number or activity of said cells.

12. The radioconjugate of claim 11, wherein said binding to uPAR results in eradication or destruction of said cells.

13. A pharmaceutical composition comprising
(a) the radioconjugate of any one of claims 1-12; and
(b) a pharmaceutically acceptable carrier or excipient.

14. A method of treating or ameliorating the symptoms of severe respiratory distress in a subject in need thereof, comprising administering an effective amount of the radioconjugate of any one of claims 1-12 or the pharmaceutical composition of claim 13.

15. The method of claim 14, wherein the severe respiratory distress is a result of bacterial sepsis.

16. The method of claim 14, wherein the severe respiratory distress is a result of an infection by a respiratory virus.

17. The method of claim 16, wherein the virus is SARS-Cov2.

18. The method of any one of claims 14-17, wherein the subject is a human

19. Use of the radioconjugate or pharmaceutical composition of any one of claims 1-13 for treating or ameliorating the symptoms of severe respiratory distress in a mammalian subject, wherein an effective amount of said radioconjugate or composition is administered to a mammalian subject with said severe respiratory distress.

20. Use of a radioconjugate of any one of claims 1-12 for the manufacture of a medicament for treatment of severe respiratory distress in a subject in need thereof.


SEQUENCE LISTING (23 sequences)

SEQ ID NO: 1 - protein, 113 aa, Artificial Sequence
  Synthetic
  misc_feature (3)..(3): Xaa can be any naturally occurring amino acid
DIXLTQSPLTLSVTIGQPASISCKSSQSLLDSDGKTYLNWLLQRPGQSPKRLIYLVSKLDSGVPDRFTGSGSGTDFTLKISRVEAEDLGVYYCWQGTHFPLTFGAGTKLELKL

SEQ ID NO: 2 - protein, 117 aa, Artificial Sequence
  Synthetic
VQLQESGPELVKTGASVKISCKASGYSFTSYYMHWVKQSHGKSLEWIGEINPYNGGASYNQKIKGRATFTVDTSSRTAYMQFNSLTSEDSAVYYCARSIYGHSVLDYWGQGTTVTVS

SEQ ID NO: 3 - protein, 16 aa, Artificial Sequence
  Synthetic
KSSQSLLDSDGKTYLN

SEQ ID NO: 4 - protein, 7 aa, Artificial Sequence
  Synthetic
LVSKLDS

SEQ ID NO: 5 - protein, 9 aa, Artificial Sequence
  Synthetic
WQGTHFPLT

SEQ ID NO: 6 - protein, 10 aa, Artificial Sequence
  Synthetic
GYSFTSYYMH

SEQ ID NO: 7 - protein, 17 aa, Artificial Sequence
  Synthetic
EINPYNGGASYNQKIKG

SEQ ID NO: 8 - protein, 10 aa, Artificial Sequence
  Synthetic
SIYGHSVLDY

SEQ ID NO: 9 - protein, 106 aa, Artificial Sequence
  Synthetic
DIVLTQSPDITAASLGQKVTITCSASSSVSYMHWYQQKSGTSPKPWIFEISKLASGVPARFSGSGSGTSYSLTISSMEAEDAAIYYCQQWNYPFTFGGGTKLEIKR

SEQ ID NO: 10 - protein, 117 aa, Artificial Sequence
  Synthetic
VKLQQSGPEVVKPGASVKISCKASGYSFTNFYIHWVKQRPGQGLEWIGWIFHGSDNTEYNEKFKDKATLTADTSSSTAYMQLSSLTSEDSAVYFCARWGPHWYFDVWGQGTTVTVSS

SEQ ID NO: 11 - protein, 10 aa, Artificial Sequence
  Synthetic
SASSSVSYMH

SEQ ID NO: 12 - protein, 7 aa, Artificial Sequence
  Synthetic
EISKLAS

SEQ ID NO: 13 - protein, 8 aa, Artificial Sequence
  Synthetic
QQWNYPFT

SEQ ID NO: 14 - protein, 10 aa, Artificial Sequence
  Synthetic
GYSFTNFYIH

SEQ ID NO: 15 - protein, 17 aa, Artificial Sequence
  Synthetic
WIFHGSDNTEYNEKFKD

SEQ ID NO: 16 - protein, 9 aa, Artificial Sequence
  Synthetic
WGPHWYFDV

SEQ ID NO: 17 - protein, 19 aa, Artificial Sequence
  Synthetic
CCTKSGCNHPDLDVQYPSG

SEQ ID NO: 18 - protein, 18 aa, Artificial Sequence
  Synthetic
CCTKSGCNHPDLDVQYPS

SEQ ID NO: 19 - protein, 17 aa, Artificial Sequence
  Synthetic
CCTKSGCNHPDLDVQYP

SEQ ID NO: 20 - protein, 16 aa, Artificial Sequence
  Synthetic
CCTKSGCNHPDLDVQY

SEQ ID NO: 21 - protein, 15 aa, Artificial Sequence
  Synthetic
CCTKSGCNHPDLDVQ

SEQ ID NO: 22 - protein, 10 aa, Artificial Sequence
  Synthetic
KSGCNHPDLD

SEQ ID NO: 23 - protein, 10 aa, Artificial Sequence
  Synthetic
KSGCNHPDLD